(19) United States
(12) Patent Application Publication
(10) Pub. No.: US 2022/0304862 A1
(43) Pub. Date: Sep. 29, 2022

(54) Method And Device For Substance Delivery To The Inner Ear

(71) Applicant: UNIVERSITY OF BRIGHTON, East Sussex (GB)

(72) Inventors: Andrei LUKASHKIN, East Sussex (GB); Natalia ZAKHAROVA, East Sussex (GB); Yuri YARIN, Dresden (DE)

(21) Appl. No.: 17/596,457

(22) PCT Filed: Jun. 10, 2020
(86) PCT No.: PCT/GB2020/051397

§ 371 Date: Dec. 10, 2021

(30) Foreign Application Priority Data
Jun. 10, 2019 (GB) 1908260.1

(51) Int. Cl. A61F 11/00; A61M 31/00

(52) U.S. Cl. CPC A61F 11/00; A61M 31/002; A61M 2037/0007

(57) ABSTRACT

The present disclosure provides a device (30) for delivering a substance to the inner ear. The device (30) comprises a pressure oscillator (32) configured to generate pressurised air at a predetermined oscillation frequency, an aural probe (31) comprising an air passageway extending from a first end of the aural probe (31) to a second end of the aural probe (31), the first end being the end to be inserted into the ear canal, a tube (33) arranged to couple the second end of the aural probe (31) to the pressure oscillator (32), and a pressure relief valve (34) disposed at the tube (33) and/or the aural probe (31) configured to release a portion or the generated pressurised air from within the tube (33) and/or the aural probe (31) when a pressure within the tube (33) and/or the aural probe (31) reaches a predetermined maximum pressure. The present disclosure further provides a method for delivering a substance to the inner ear, the method comprising the steps of: (i) administering the substance to the middle ear; and (ii) generating oscillating air pressure within the ear canal.

The present invention relates to methods and devices for delivering substances to the inner ear of a mammal.

### BACKGROUND TO THE PRESENT INVENTION

The mammalian inner ear is one of the least accessible areas of the body for drug delivery. Systemic administration of many drugs, for example frequently used corticosteroids and aminoglycoside antibiotics, is severely limited by the blood-labyrinth barrier. Administration via the ear canal is ineffective due to the impermeable tympanic membrane (ear drum). Administration directly to the cochlea is highly invasive, requiring surgical intervention. Current methods therefore involve local intratympanic administration, i.e. administration to the tympanic cavity in the middle ear. For many existing and newly emerging classes of drugs and therapies, including local anaesthetics, antioxidants, apoptosis inhibitors, neurotransmitters and their antagonists, monoclonal antibodies, growth factors, signalling pathway regulators and genetic material, local intratympanic delivery may be the only option.

Intratympanic administration typically involves injection through the tympanic membrane into the tympanic cavity. This is a minimally invasive, routine procedure. However, retention of administered substances in the tympanic cavity is not straightforwardly achieved. The Eustachian tube connects the tympanic cavity to the nasopharynx, leading to the relatively rapid clearance of administered substances from the tympanic cavity.

The inner ear comprises the cochlea, the anatomy of which provides particular difficulties for the delivery of drugs and other substances thereto. The cochlea is a bony, coiled tube comprising three fluid filled compartments. The upper and lower compartments (the scala vestibuli and the scala tympani, respectively) form the cochlear spiral and communicate through direct connection at the helicotrema, and comprise a fluid: perilymph. The interfaces between the cochlea and tympanic cavity are the round and oval windows; semi-permeable membranes of only small surface area (the surface area of the larger window, the round window, is only 2.2 mm² in humans).

Substances that permeate the round or oval window are not sufficiently and/or uniformly distributed along the length of the cochlear spiral. The extent to which a substance is distributed in the scala tympani and scala vestibuli is limited by the low flow rate of perilymph within the cochlea. The longitudinal flow of perilymph in the cochlea is relatively slow, if present at all, and as such substance distribution in the perilymph is reliant on mere passive diffusion. Furthermore, passive diffusion along the scala tympani is constrained by the geometry of the cochlea, which comprises a long and narrow tube having a gradually decreasing cross-section from the round window at the base to the apex. This creates large base to apex concentration gradients within the cochlea.

Since the cochlear apex is where human speech processing is initiated, it is where substance delivery to the cochlea has the greatest potential therapeutic and socioeconomic impact. Direct measurements of the distribution of marker ions, corticosteroids and antibiotics, or measurements of the physiological effects caused by the diffusion of drugs into the cochlea have demonstrated that the concentration of substances applied to the round window is much higher at the cochlear base than at the apex. Allowing a substance to remain in contact with the round window for an arbitrarily long time has been found to have no significant effect on the large base-to-apex concentration gradients. Long residency of a substance at the round window has been found only to lead to the establishment of a steady-state base-to-apex gradient of substance concentration, with the magnitudes dependent on the properties of the specific substance.

It is therefore desirable to provide an improved method and device for delivering and distributing substances to the inner ear, particularly to the cochlea and particularly along the cochlear spiral.

### SUMMARY

According to an aspect of the present invention, there is provided a device for assisting in distributing a substance in the inner ear, comprising: a pressure oscillator; an aural probe having an end insertable in an ear canal; and a fluid passageway fluidly communicating the pressure oscillator and the end of the aural probe insertable in the ear canal; wherein the pressure oscillator is configured to generate an oscillating fluid pressure change in the fluid passageway.

The pressure oscillator may be configured to generate both positive and negative pressures in the fluid passageway.

The pressure oscillator may be configured to generate a biphasic oscillating pressure change in the fluid passageway.

The predetermined maximum positive and/or negative operating pressure may be between 0.5 kPa and 20 kPa, and preferably between 0.5 kPa and 4 kPa, and more preferably between 0.5 kPa and 2 kPa.

The maximum amplitude of the operating pressure may be 4 kPa, and, optionally 2 kPa. Such an arrangement allows the ear drum to be driven to its extreme positions even when the middle ear cavity is filled with a liquid. The pressure oscillator may be configured to generate air pressure oscillations at a frequency in the range of 1 to 20 Hz, and optionally 2 to 20 Hz, and optionally 4 to 18 Hz, and optionally 8 to 16 Hz.

The device may comprise a pressure relief valve communicating with the fluid passageway and configured to open when a pressure within the fluid passageway reaches a predetermined maximum positive and/or negative pressure.

According to an aspect of the present invention, there is provided a method of operating a device for assisting in distributing a substance in the inner ear, the device including an aural probe having an end insertable in an ear canal, the method comprising generating an oscillating fluid pressure change in a fluid passageway of the device to supply both positive and negative pressures at an end of an aural probe.

The device may be used to assist in delivering a substance to the inner ear, wherein the substance has been administered to the middle ear and wherein the device generates an oscillating air pressure within the ear canal. Reference in the application to the device delivering a substance corresponds to the device assisting in delivering a substance within the inner ear that has previously been administered to the middle ear. This may include at least one of inducing or promoting transfer of a substance from the middle ear to the inner ear, and inducing distribution of inducing or promoting distribution of a substance which is in the inner ear along the cochlear spiral.

According to an aspect of the present invention, there is provided a computer program product comprising instructions which, when the program is executed by a processor, cause the processor to carry out at least one of the methods described above.

According to an aspect of the present invention, there is provided a computer readable medium having stored therein the computer program product as set out above.

According to an aspect of the present invention, there is provided a non-transitory computer readable medium comprising computer readable instructions that, when executed by a processor, cause performance of the method as set out above.

In an aspect the present invention provides a device for delivering a substance to the inner ear, comprising a pressure oscillator configured to generate pressurised air at a predetermined oscillation frequency;

an aural probe comprising an air passageway extending from a first end of the aural probe to a second end of the aural probe, the first end being the end to be inserted into the ear canal;

a tube arranged to couple the second end of the aural probe to the pressure oscillator; and a pressure relief valve disposed at the tube and/or the aural probe configured to release a portion of the pressurised air from within the tube and/or the aural probe when the pressure within the tube and/or the aural probe reaches a predetermined maximum pressure.

According to embodiments of the present invention, the aural probe may be inserted into e.g. the ear canal and a mammalian subject such as a human patient, and the pressure oscillator may be operated such that pressurised air set at a predetermined frequency of oscillation may be generated. The pressurised air is delivered from the pressure oscillator to the aural probe, and the oscillating pressurised air causes reciprocating movement of the round and/or oval window membrane and/or stapes, leading to perilymph flow. The pressurised air is delivered from the pressure oscillator to the aural probe via a fluid passageway, which may be provided by a tube. As a result of the movement of the round and/or oval window membrane and/or stapes induced by the device according to preferred embodiments, once substances have been deposited in the middle ear and diffused into the cochlear perilymph, it is possible to improve the delivery of the substances to the inner ear, along the cochlear spiral, and may improve delivery to the cochlear apex. Distribution of the substances to the inner ear, along the cochlear spiral may be improved. The pressure relief valve may be operated to release some of the pressure from within the tube and/or the aural probe if and when the pressure within the tube and/or aural probe approaches or exceeds a predetermined maximum amplitude (e.g. 4 kPa). According to embodiments, the pressure relief valve ensures that the patient would not experience discomfort and that the patient's tympanic membrane and inner ear are not injured or otherwise damaged due to excessive pressure, it is therefore possible to safely use a range of pressure up to the predetermined maximum pressure on the subject.

In an embodiment, the device may further comprise a pressure sensor coupled to the tube configured to determine the pressure within the tube. The pressure sensor may be at the aural probe or the pressure oscillator. The pressure sensor may be configured to determine an instantaneous pressure and/or a time-averaged pressure within the tube, as desired.

In an embodiment, the device may further comprise a feedback module coupled to the pressure sensor and the pressure oscillator, the feedback module being configured to compare the pressure within the fluid passageway, for example the tube, determined by the pressure sensor with a predetermined operating pressure range, and when it is determined that the pressure within the tube is outside of the predetermined operating pressure range, to cause the pressure oscillator to adjust when generating pressurised air.

Alternatively or additionally, embodiments of the device may further comprise a feedback module coupled to the pressure sensor and the pressure relief valve, the feedback module being configured to compare the pressure within the fluid passageway, for example the tube, determined by the pressure sensor with the predetermined maximum pressure, and when it is determined that the pressure within the tube is outside of the predetermined maximum pressure, to cause the pressure relief valve to release pressurised air within the tube and/or the aural probe.

In embodiments, the feedback module may be further configured to perform the comparison continuously, intermittently or periodically and adjust the pressure within the tube based on the comparison. It is therefore possible to monitor and maintain the pressure experienced by the patient at a safe level.

According to embodiments, the device can provide the beneficial technical effects described herein at any suitable and desirable pressure and frequency range. However, in an embodiment, the predetermined operating pressure range of pressure amplitude may be set between 0.5 kPa and 4 kPa, and/or the predetermined maximum pressure amplitude may be set at 4 kPa to ensure patients' comfort and safety, whilst providing maximum efficiency.

In an embodiment, the aural probe may comprise an auxiliary air passageway extending from an interior surface of the air passageway to an exterior surface of the aural probe at the second end, the auxiliary air passageway having a valve for closing or opening the auxiliary air passageway. According to the embodiment, the valve of the auxiliary air passageway may be opened once the aural probe is inserted into and sealed within the patient's ear to equalise the pressure on either side of the tympanic membrane.

In an embodiment, the aural probe may comprise a Siegel speculum or pneumatic otoscope arranged for visual inspection of the tympanic membrane movement. This may be particularly useful when the patient is less able or unable to provide adequate feedback, for example if the patient is a child or under anaesthetics.

In an embodiment, the aural probe may comprise a seal for sealing the ear canal when the aural probe is inserted into the ear canal. In some embodiments, the seal may comprise a disposal tip for improved convenience or hygiene.

In a further aspect, the present invention provides a method for delivering a substance to the inner ear, the method comprising the steps of:
i) administering the substance to the middle ear; and
ii) generating oscillating air pressure within the ear canal.

The invention also provides a method for delivering a substance to the inner ear, wherein the substance has been administered to the middle ear and wherein the method comprises generating oscillating air pressure within the ear canal.

Alternatively viewed, the present invention provides a method for assisting in the delivery of a substance to the inner ear, the method comprising the steps of:
i) administering the substance to the middle ear; and
ii) generating oscillating air pressure within the ear canal.

The invention also provides a method for assisting in the delivery of a substance to the inner ear, wherein the substance has been administered to the middle ear and wherein the method comprises generating oscillating air pressure within the ear canal.

Alternatively viewed, the present invention provides a method of assisting in distributing a substance in the inner, the method comprising the steps of:
i) administering the substance to the middle ear; and
ii) generating oscillating air pressure within the ear canal.

The invention also provides a method of assisting in distributing a substance in the inner ear, wherein the substance has been administered to the middle ear and wherein the method comprises generating oscillating air pressure within the ear canal.

In a further aspect, the present invention provides a method for inducing or promoting transfer of a substance from the middle ear to the inner ear, the method comprising the steps of:
i) administering the substance to the middle ear; and
ii) generating oscillating air pressure within the ear canal.

The invention also provides a method for inducing or promoting transfer of a substance from the middle ear to the inner ear, wherein the substance has been administered to the middle ear and wherein the method comprises generating oscillating air pressure within the ear canal.

In a further aspect, the present invention provides a method for distributing a substance along the cochlear spiral, the method comprising the steps of:
i) administering the substance to the middle ear; and
ii) generating oscillating air pressure within the ear canal.

The invention also provides a method for distributing a substance along the cochlear spiral, wherein the substance has been administered to the middle ear and wherein the method comprises generating oscillating air pressure within the ear canal.

The terms delivering and distributing are used interchangeably throughout.

The invention also provides a method for inducing or promoting periodic flow of perilymph within the inner ear, the method comprising generating oscillating air pressure within the ear canal.

In a further aspect, the present invention provides a method of treating or preventing an inner ear disease, the method comprising the steps of:
i) administering a therapeutic compound to the middle ear; and
ii) generating oscillating air pressure within the ear canal.

The present invention also provides a method of treating or preventing an inner ear disease, wherein a therapeutic compound has been administered to the middle ear and wherein the method comprises generating oscillating air pressure within the ear canal.

Alternatively viewed, the present invention provides a therapeutic compound for use in a method of treating or preventing an inner ear disease, wherein said method comprises the steps of:
i) administering the therapeutic compound to the middle ear; and
ii) generating oscillating air pressure within the ear canal.

The present invention also provides a therapeutic compound for use in a method of treating or preventing an inner ear disease, wherein the therapeutic compound has been administered to the middle ear and wherein said method comprises generating oscillating air pressure within the ear canal.

Alternatively viewed, the present invention provides the use of a therapeutic compound in a method of treating or preventing an inner ear disease, wherein said method comprises the steps of:
i) administering the therapeutic compound to the middle ear; and
ii) generating oscillating air pressure within the ear canal.

The present invention also provides the use of a therapeutic compound in a method of treating or preventing an inner ear disease, wherein the therapeutic compound has been administered to the middle ear and wherein said method comprises generating oscillating air pressure within the ear canal.

Alternatively viewed, the present invention provides a therapeutic compound for use in the manufacture of a medicament for use in the treatment or prevention of an inner ear disease, wherein said treatment or prevention comprises the steps of:
i) administering the therapeutic compound to the middle ear; and
ii) generating oscillating air pressure within the ear canal.

In all methods and uses of the present invention, the generation of oscillating air pressure is preferably achieved via the use of a device of the invention. Reference herein to a device of the invention is a reference to any device described herein.

Thus, in a further aspect, the present invention also provides the use of a device of the invention in a method of the invention. The method may be any method described herein.

Alternatively viewed, the present invention provides the following uses:

Use of a device of the invention to generate oscillating air pressure within the ear canal.

Use of a device of the invention to deliver a substance to the inner ear, the use comprising:
i) administering the substance to the middle ear;
ii) inserting the aural probe of the device into the ear canal; and
iii) operating the device to generate oscillating air pressure within the ear canal.

Use of a device of the invention to deliver a substance to the inner ear, wherein the substance has been administered to the middle ear and wherein the use comprises inserting the aural probe of the device into the ear canal and operating the device to generate oscillating air pressure within the ear canal.

Use of a device of the invention to assist in the delivery of a substance to the inner ear, the use comprising:
i) administering the substance to the middle ear;
ii) inserting the aural probe of the device into the ear canal; and
iii) operating the device to generate oscillating air pressure within the ear canal.

Use of a device of the invention to assist in the delivery of a substance to the inner ear, wherein the substance has been administered to the middle ear and wherein the use comprises inserting the aural probe of the device into the ear canal and operating the device to generate oscillating air pressure within the ear canal

Use of a device of the invention to assist in distributing a substance in the inner, the use comprising:
i) administering the substance to the middle ear;
ii) inserting the aural probe of the device into the ear canal; and
iii) operating the device to generate oscillating air pressure within the ear canal.

Use of a device of the invention to assist in distributing a substance in the inner ear, wherein the substance has been administered to the middle ear and wherein the use comprises inserting the aural probe of the device into the ear canal and operating the device to generate oscillating air pressure within the ear canal

Use of a device of the invention to induce or promote transfer of a substance from the middle ear to the inner ear, the use comprising:
i) administering the substance to the middle ear;
ii) inserting the aural probe of the device into the ear canal; and
iii) operating the device to generate oscillating air pressure within the ear canal.

Use of a device of the invention to induce or promote transfer of a substance from the middle ear to the inner ear, wherein the substance has been administered to the middle ear and wherein the use comprises inserting the aural probe of the device into the ear canal and operating the device to generate oscillating air pressure within the ear canal.

Use of a device of the invention to distribute a substance along the cochlear spiral, the use comprising:
i) administering the substance to the middle ear;
ii) inserting the aural probe of the device into the ear canal; and
iii) operating the device to generate oscillating air pressure within the ear canal.

Use of a device of the invention to distribute a substance along the cochlear spiral, wherein the substance has been administered to the middle ear and wherein the use comprises inserting the aural probe of the device into the ear canal and operating the device to generate oscillating air pressure within the ear canal.

Use of a device of the invention to induce or promote periodic flow of perilymph within the inner ear, wherein the use comprises inserting the aural probe of the device into the ear canal and operating the device to generate oscillating air pressure within the ear canal.

Use of a device of the invention to treat or prevent an inner ear disease, the use comprising the steps of:
i) administering a therapeutic compound to the middle ear;
ii) inserting the aural probe of the device into the ear canal; and
iii) operating the device to generate oscillating air pressure within the ear canal.

Use of a device of the invention to treat or prevent an inner ear disease, wherein a therapeutic compound has been administered to the middle ear and wherein the use comprises inserting the aural probe of the device into the ear canal and operating the device to generate oscillating air pressure within the ear canal.

In the uses of the invention, the device may be as defined anywhere herein.

### BRIEF DESCRIPTION OF THE DRAWINGS

Various embodiments of the present invention will now be described, by way of example only, and with reference to the accompanying drawings in which:

FIG. 1 shows a schematic of the mammalian hearing organ, showing the ear canal (11), tympanic membrane (12), Eustachian tube (13), tympanic cavity (14), maleus (15), incus (16), stapes (17), oval window (18), round window (19), scala vestibuli (20), scala tympani (21) and helicotrema (22).

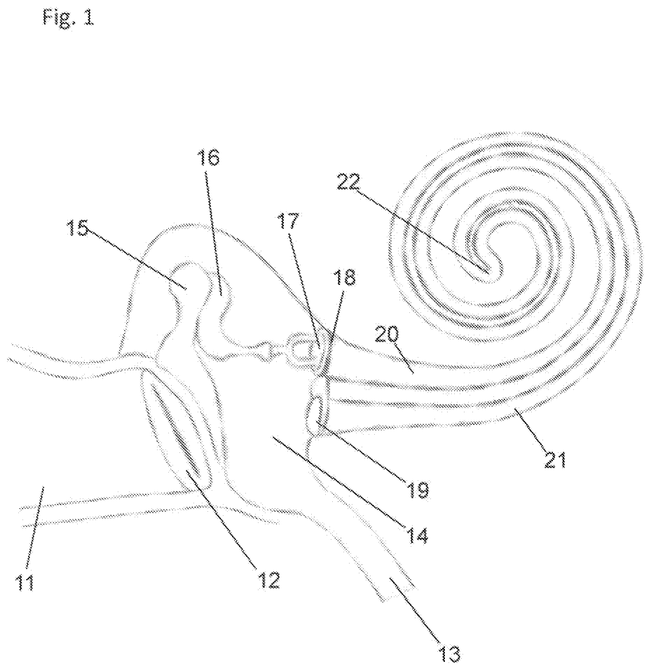

FIG. 2 shows representative examples of elevation of the auditory nerve pure tone threshold responses when salicylate diffuses through the perilymph passively (A) or is redistributed via the generation of oscillating pressure in the ear canal (B). 5 μl of 100 mM salicylate solution was applied to the round window at time=0. Salicylate was washed out after 80 minutes (A) and 74 minutes (B) of application. (B) Pressure oscillations at 4 Hz were applied to the ear canal during 5 minutes before each non-zero time point plotted and the auditory nerve thresholds were measure during following 5-minute interval without pressure oscillations. Similar results were observed in four further preparations.

FIG. 3 is a schematic drawing of a device for delivering substances to the cochlea according to an embodiment.

FIG. 4 shows the differing elevation of the auditory nerve threshold responses following no treatment (passive diffusion, crosses) or generation of oscillating pressure within the ear canal (oscillating pressure, circles) in response to pure tones of frequency 27 kHz (A), 21 kHz (B), 15 kHz (C), 11 kHz (D), 5 kHz (E), 3 kHz (F) and 1 kHz (G). The experimental protocols were the same as in FIG. 2. Pooled data for 5 experiments for each experimental protocol.

### DETAILED DESCRIPTION

In one aspect, the present invention provides a method for delivering a substance to the inner ear, the method comprising the steps of:
i) administering the substance to the middle ear; and
ii) generating oscillating air pressure within the ear canal.

This method is viewed alternatively as a method for assisting in the delivery of a substance to the inner ear, or a method for assisting in distributing a substance in the inner.

The inner ear comprises the cochlea, the vestibular organ and the auditory nerve. Delivery of a substance to the inner ear in accordance with the invention may be delivery to any of these sites. Preferably however, delivery is to the cochlea. The membraneous barriers between the middle ear and the cochlea are the round and oval windows. The two windows separate the middle ear (tympanic cavity) from the scala tympani and scala vestibuli of the cochlea. The scala tympani and scala vestibuli are filled with a fluid; perilymph. The scala tympani and scala vestibuli form the cochlear spiral, coiling and narrowing towards the cochlear apex, where they communicate through the helicotrema. The cochlea therefore comprises the cochlear base (close to the round and oval windows) and a narrower cochlear apex. Delivery to the cochlea in accordance with the present invention may be delivery to any of these cochlear sites. Preferably, delivery is to the scala tympani and scala vestibuli. Preferably delivery to the inner ear is delivery along the cochlear spiral. Preferably delivery is to the cochlear apex.

Current methods of delivering substances to the inner ear do not adequately deliver substances along the cochlear spiral. Due to the geometry of the cochlea and the slow rate of perilymph flow within the cochlea, substances delivered to the middle ear are not delivered (i.e. distributed) along the cochlear spiral to a satisfactory extent by passive diffusion. The methods and devices of the present invention achieve delivery along the cochlear spiral, even to the cochlear apex.

The cochlear spiral comprises the cochlear base adjacent to the round and oval windows and the cochlear apex which is the most distal point from the round and oval windows. The base and apex can therefore be considered as point locations; the extremities defining the cochlear spiral length. However, as used herein in the context of delivery of substances, the term “cochlear base” is the region of the cochlear spiral most proximal to the round and oval windows, said region comprising 25% of the cochlear spiral length. In other words, in the context of delivery of substances, the cochlear base is the basal region of the cochlear spiral, said region comprising 25% of the cochlear spiral length, alternatively termed the basal quarter of the cochlear spiral. Similarly, as used herein in the context of delivery of substances, the term “cochlear apex” is the region of the cochlear spiral most distal from the round and oval windows (i.e. most proximal to the apex), said region comprising 25% of the cochlear spiral length. In other words, in the context of delivery of substances, the cochlear apex is the apical region of the cochlear spiral, said region comprising 25% of the cochlear spiral length, alternatively termed the apical quarter of the cochlear spiral. The middle region of the cochlear spiral is that between the basal and apical regions, said region comprising 50% of the cochlear spiral length. The basal half of the cochlear spiral is 50% of the cochlear spiral length proximal to the round and oval windows. The apical half of the cochlear spiral is 50% of the cochlear spiral distal from the round and oval windows (i.e. proximal to the apex).

As used herein, the term “along the cochlear spiral” encompasses delivery not only to the basal region/quarter of the cochlear spiral but also to the middle region of the cochlear spiral, and preferably also to the apical region/quarter of the cochlear spiral. Delivery is preferably to the apical half of the cochlear spiral, preferably to the apical quarter of the cochlear spiral. The delivery of the substance is preferably uniform distribution of the substance.

As explained above, using current delivery methods, passive diffusion of substances within the cochlear spiral is limited, with significant base to apex concentration gradients being established. The methods of the present invention overcome such problems, resulting in the reduction in such concentration gradients. The difference in the concentration of a substance between a first point more proximal to the cochlear base and a second point more proximal to the cochlear apex is reduced using the methods of the present invention as compared to the difference in concentration observed with the same substance and between the same two points in the absence of the performance of the step of generating oscillating air pressure within the ear canal.

The external ear comprises the pinna and the ear canal (also known as the auditory canal). The tympanic membrane (ear drum) creates a barrier with the middle ear. The middle ear comprises the tympanic cavity (including the ossicular chain formed by the malleus, the incus and the stapes) and the Eustachian tube. Administration of a substance to the middle ear in accordance with the present invention may be administration to any of these sites. For the purposes of the present invention, administration to the round and oval windows (the membraneous barriers between the middle ear and the inner ear) is considered administration to the middle ear, since it is the tympanic cavity side of the window to which administration is made. Preferably, administration is to the tympanic cavity or to the round or oval window. Preferably administration is to the tympanic cavity or to the round window.

Administration to the middle ear is known as intratympanic administration. Methods of administering substances to the middle ear, particularly to the tympanic cavity, are well-known in the art and any such method may be used in the methods of the present invention. Preferred methods include transtympanic injection, administration via an intratympanic device and administration via the Eustachian tube.

A preferred method of administration to the middle ear is transtympanic injection, which is a straightforward, low-risk, outpatient procedure performed under local anaesthetic. In such procedures, the tympanic membrane is perforated using a fine needle (preferably 22 to 25 G in humans, 27 to 30 G in animals) and a volume of solution (preferably 0.4-0.6 mL in humans, 50-200 μL in animals) is injected, preferably to fill the middle ear cavity. Injection may be performed using an otendoscopy device comprising a fiber optic lens for viewing and two working channels: a catheter channel equipped with a fine needle for delivering drugs and a suction channel for removing adhesions when the round or oval window are obstructed.

Alternatively, administration to the middle ear may be performed by an intratympanic administration device, preferably selected from the group consisting of a microwick, a microcatheter, a biodegradable polymer device and a non-biodegradable polymer device. Microwicks comprise an absorbent wick, which is inserted through a ventilation tube in the tympanic membrane. The wick carries solutions administered to the external ear canal into the middle ear. Microcatheters comprise two lumens, one for administration to the middle ear and one for withdrawal of fluid. The microcatheter is inserted via a tympanomeatal flap, and the end of the catheter exits from the outer ear canal, allowing connection to pumping systems for the infusion of drugs, e.g. osmotic pumps and micropumps. Polymeric-based devices are solid sponges, beads or discs soaked in a drug solution and placed into the middle ear via surgery. Preferably the polymeric-based device is biodegradable, avoiding the need for surgical removal. Biodegradable materials used in such devices include gelfoam (sterile compressed sponge comprised of purified porcine gelatin) and separapack (carboxymethylcellulose and hyaluronic acid discs). Preferably, any administration device is removed prior to the generation of oscillating air pressure within the ear canal.

Alternatively, administration to the middle ear may be achieved via the Eustachian tube. This typically comprises using intranasal medication, preferably drops or sprays, more preferably drops. Administration via the Eustachian tube orifice may also comprise cannulation thereof.

The invention also provides a method for delivering a substance to the inner ear (i.e. for assisting in the delivery of a substance to the inner ear, or for assisting in distributing a substance in the inner), and other methods, wherein the substance has already been administered to the middle ear and wherein the method comprises generating oscillating air pressure within the ear canal. Such methods of the invention do not comprise the active step of administering the substance to the middle ear; this has been done previously so the substance is already in situ in the middle ear prior to the commencement of the method of the invention.

The methods of the present invention are preferably performed on a human or mammalian animal subject, most preferably a human subject. Thus, the features of the ear referred to herein are preferably features of the human or mammalian animal ear, most preferably the human ear.

The substance delivered according to the present invention may be any substance capable of permeating the round and/or oval window membranes. Such compounds are well-known and could be readily identified by the skilled person. The round and oval window membrane permeability is influenced by different factors such as size, concentration, hydrophilicity and electrical charge of the substance as well as by the thickness and condition of the window membrane. It is within the competencies of one of ordinary skill in the art to determine the ability of a substance of interest to permeate the round and/or oval window membrane. The permeability of the round window membrane is discussed for instance in Goycoolea & Lundman, 1997, Microsc Res Tech. 36(3):201-11 and Goycoolea, 2001, Acta Otolaryngol. 121(4): 437-447.

Preferably, the delivered substance is a therapeutic compound. The term “therapeutic compound” as used herein refers to any physical, chemical or biological substance which may be used in the treatment, cure, prophylaxis, prevention, or diagnosis of a pathological condition, e.g. a disease or disorder, or which may be used to otherwise enhance physical, psychical or mental well-being. The terms “therapeutic compound”, “active agent” and “drug” are used interchangeably herein.

However, the delivered substance may be selected from the group consisting of a therapeutic compound, an adjuvant, a biologically inert compound, a dye, an imaging compound (e.g. a contrast agent), a cosmetic agent, a diagnostic agent and a physiologically acceptable excipient. Preferably the physiologically acceptable excipient is selected from the group consisting of a stabilizing agent, an osmotic agent, a preservative, a tonicity agent, a chelating agent, a mucoadhesive agent and a permeation enhancing agent. The delivered substance may be a mixture of two or more such substances. Preferred dyes include rhodamine B, carboxycyanine and nile red. A preferred contrast agent is gadolinium.

Preferably, the therapeutic compound is selected from the group consisting of antimicrobial agents (including antibacterial, antiviral agents and anti-fungal agents), anti-tumor agents, thrombin inhibitors, antithrombogenic agents, thrombolytic agents, fibrinolytic agents, vasospasm inhibitors, calcium channel blockers, vasodilators, antihypertensive agents, antibiotics, inhibitors of surface glycoprotein receptors, antiplatelet agents, antimitotics, microtubule inhibitors, anti-secretory agents, actin inhibitors, remodeling inhibitors, antimetabolites, antiproliferatives (including anti-angiogenesis agents), anticancer chemotherapeutic agents, anti-inflammatory steroid or nonsteroidal anti-inflammatory agents, immunosuppressive agents, growth hormone antagonists, growth factors, dopamine agonists, radiotherapeutic agents, extracellular matrix components, ACE inhibitors, ROS scavengers and their inducers, chelators, antioxidants, anti-polymerases, photodynamic therapy agents, prostaglandin receptor agonists, inhibitors of the JNK stress kinase, peroxisome proliferator-activated receptor agonists, NOX and NOS enzymes inhibitors, RNAi agents, glutamate receptor blocker, glutathione peroxidase inducers, anti-apoptotic agents, differentiation modulating agents and generic material (plasmids and viral vectors).

Preferably, the substance is selected from the group consisting of an antibiotic, an antiseptic, a local anaesthetic, a dye, a protein, genetic material, a virus or a nanoparticle.

Preferably the therapeutic compound is a therapeutic compound for treating an inner ear disease. Preferably the therapeutic compound is selected from the group consisting of a protein, a peptide, a non-protein drug, a small chemical molecule, a carbohydrate, a lipid, a lipopolysaccharide, a nucleic acid, a viral vector and a stem cell.

Preferably the therapeutic compound is selected from the group consisting of a steroid, an antibiotic, an antioxidant, an anaesthetic, an n-Methyl-d-aspartate receptor antagonist, an antiviral agent, an antifungal agent, an apoptosis inhibitor, an enzyme inhibitor, a neurotrophic or neuroprotective agent, an antibody, a nucleic acid, a viral vector or a stem cell.

Preferably the steroid is a corticosteroid, preferably dexamethasone or methyl prednisolone. Preferably the antibiotic is an aminoglycoside antibiotic, preferably gentamycin, streptomycin or kanamycin. Preferably the antioxidant is n-acetyl cysteine, d-methionine or thiourea. Preferably the anaesthetic is a local anaesthetic, preferably lidocaine. Preferably the n-methyl-d-aspartate receptor antagonist is esketamine, gacyclidine or ifenprodil. Preferably the antiviral agent is cidofovir or ganciclovir. Preferably the apoptosis inhibitor is a JNK inhibitor, preferably AM-111 peptide. Preferably the neurotrophin is BDNF, neurotrophic factor-3 or a growth factor. Preferably the antibody is a monoclonal antibody, preferably Infliximab, Golimumab, Anakinra or Rituximab. Preferably the nucleic acid is a polynucleotide, antisense nucleotide, RNA molecule, DNA molecule, siRNA molecule, miRNA molecule, gene or vector. Preferably the vector is a plasmid or viral vector, preferably a plasmid. Preferably the nucleic acid is for gene therapy.

The administered substance, e.g. the therapeutic compound, is preferably administered as a pharmaceutically acceptable salt, solvate, clathrate, prodrug, tautomer or a metabolic derivative thereof.

The administered substance, e.g. the therapeutic compound, is preferably in the form of a pharmaceutical composition also comprising one or more pharmaceutically acceptable diluents, carriers or excipients. The pharmaceutical compositions may be formulated in any convenient manner according to techniques and procedures known in the pharmaceutical art. “Pharmaceutically acceptable” as used herein refers to ingredients that are compatible with other ingredients of the compositions as well as physiologically acceptable to the recipient. The nature of the composition and carriers or excipient materials, dosages etc. may be selected in routine manner according to choice and the desired route of administration, purpose of treatment etc. Dosages may likewise be determined in routine manner and may depend upon the nature of the molecule, purpose of treatment, age of patient, mode of administration etc.

Preferably, the excipient is selected from the group consisting of a stabilizing agent, an osmotic agent, a preservative, a tonicity agent, a chelating agent, a mucoadhesive agent and a permeation enhancing agent. Preferably the therapeutic compound is the form of a pharmacologically acceptable salt.

Preferably, the substance, e.g. the therapeutic compound, is administered in combination with one or more accessory compounds selected from the group consisting of a further therapeutic compound, an adjuvant, a biologically inert compound, a dye or an imaging compound (e.g. a contrast agent), a cosmetic agent and a diagnostic agent.

The administered substance is preferably in the form of or comprised within a liquid, hydrogel or nanovector, most preferably a liquid. Preferably the liquid is an aqueous solution or a lipid-based formulation.

Hydrogels have a high viscosity and viscoelastic properties that decreases the rate of flow through the Eustachian tube thus allowing longer residence times in the middle ear. Hydrogels of use in the present invention preferably comprise in addition to the substance to be delivered, synthetic thermosensitive polymers, preferably poloxamer 407, PLGA-PEG-PLGA copolymer or derivatives thereof; or natural polymers, preferably chitosan, hyaluronic acid, collagen, gelatin or derivatives thereof. The preparation of hydrogels is well-known in the field and the skilled person would readily be able to prepare hydrogels comprising a substance of interest for administration to the middle ear.

Nanovectors suitable for administration to the middle ear are also well-known in the field (see for example Kechai et al., (2015) International Journal of Pharmaceutics 494: 83-101) and any such nanovector may be used in the methods of the present invention.

Preferably the nanovectors are selected from the group consisting of liposomes, lipid nanocapsules, glycerol monooleate-based nanoparticles, poly(lactic-co-glycolic acid) nanoparticles, polymerosomes and dendrimer-based nanoparticles. Polymersomes of use in the present invention include self-assembling amphiphilic block copolymers such as poly(ethyleneglycol)-b-poly(e-caprolactone) (PEG-b-PCL) and poly(2-hydroxyethylaspartamide (PHEA). Dendrimer-based nanoparticles are randomly branched dendritic polymers. Preferred dendrimer-based nanoparticles include hyperbranched poly-L-lysine (HBPL).

Preferably, the nanovectors have a size ranging from 10 to 640 nm in diameter, preferably 10 to 250 nm in diameter. Preferably, the nanovectors comprise surface modification, preferably with PEG, antibodies, peptides, carbohydrates, chitosan, hyaluronic acid, synthetic cationic lipids or folic acid.

The methods of the present invention all comprise the step of generating oscillating air pressure within the ear canal. The ear canal, also called the auditory canal, the external ear canal, the external auditory canal, the external acoustic meatus and the external auditory meatus (EAM) is the pathway extending from the pinna (external ear) to the tympanic membrane (eardrum).

The resting pressure within the ear canal is equal to the environmental pressure in mammals, including human adults and children. The resting pressure within the tympanic cavity is identical to that in the ear canal; such equilibration is one function of the Eustachian tube.

The methods of the present invention therefore comprise the step of generating pressurised air at a predetermined oscillation frequency. The means for performing such a step are preferably as described elsewhere herein. In order to generate changes in air pressure within the ear canal, the ear canal is first preferably sealed to prevent air escaping. This is readily achieved by inserting a tip of a probe in to the ear canal. Such steps are preferred in the methods of the present invention. Air pressure oscillations can then be generated by the use of a pressure pump connected to the probe. This would be within the competencies of the person of ordinary skill in the field.

When an aural probe is inserted or pushed into the ear canal, the action of the inserting or pushing may cause the build-up of static positive pressure in the region between the probe and tympanic membrane. In some embodiments of the devices, a closable (e.g. by means of a valve) auxiliary air passageway is provided through the probe, which when operated (e.g. the valve is opened) allows the pressures on either side of the tympanic membrane to be equalised after location of the probe and before operation of the device. The methods of the present invention therefore comprise a step of equalizing the air pressure within the ear canal and the air pressure within the tympanic cavity after insertion of the aural probe into the ear canal and before the generation of oscillating air pressure.

Oscillating or reciprocating pressure changes can then be generated by any means known in the art. Suitable pressure generating devices known in the art may be employed as the pressure oscillator of the device as desired and coupled to the aural probe. For instance, oscillating pressure may be generated by reciprocal displacement of a moving part within a pressure generating device, for instance by displacement of a diaphragm or membrane. Alternatively, oscillating pressure may be generated by modulation of pressure from a constant pressure source within a pressure generating device. The step of generating oscillating pressure within the ear canal therefore comprises operation of a pressure generating device to generate said oscillating pressure and this would be within the competencies of the person of ordinary skill in the art.

Typically, the oscillating pressure is generated via a pressure pump connected or otherwise coupled to the aural probe. The aural probe preferably comprises an air passageway extending from a first end, which is inserted into the ear canal and a second end. Typically, the pressure generating device, e.g. a pressure pump or oscillator, is connected to the second end of the aural probe, preferably via a tube.

Tympanometers are devices known in the art which induce pressure changes in the sealed ear canal via a pressure pump. Tympanometers are known in the field for use in testing middle ear function. They generate sound and measure reflectance of this sound from the tympanic membrane during a single cycle of very slow pressure change in the ear canal.

Air pressure oscillations may be provided by a pressure pump being coupled to a pressure oscillator, which can be configured to generate or otherwise output pressurized air at a predetermined oscillation frequency. In some embodiments, the pressure oscillator may comprise a multiplexor for alternately switching at a predetermined frequency the input to the aural probe (e.g. through the tube) between atmospheric pressure (i.e. to apply zero differential pressure across the tympanic membrane) and the pressure pump set to generate air pressure at a predetermined pressure, thus generating pressure pulses at a predetermined oscillation frequency at a predetermined magnitude.

By “oscillating air pressure” is meant that pressure is generated within the ear canal that oscillates between a maximum and a minimum pressure. The pressure generated within the ear canal corresponds to the pressure generated in the device. Pressure changes applied in the ear canal correspond to pressure changes applied in the device. A fluid passageway is defined in the device between the pressure generating device and a port of the device which communicates with the ear canal. The generated pressure changes may be monophasic (i.e. deviating from the resting pressure in a positive or negative direction) or biphasic (i.e. deviating from the resting pressure in both positive and negative directions). In other words, the maximum positive pressure generated is greater than the resting pressure and/or the maximum negative pressure generated is lower than the resting pressure. The generated pressure changes may be biphasic. In other words, the methods and uses may comprise a step of generating biphasic oscillating air pressure within the ear canal. In some embodiments, the deviation from the resting pressure in both positive and negative directions is sinusoidal.

In all cases, the maximum deviation from the resting pressure, i.e. the maximum magnitude (i.e. amplitude) of the pressure change generated within the ear canal should not exceed 20 kPa, i.e. ±20 kPa, and preferably, should not exceed 4 kPa, i.e. ±4 kPa and more preferably 2 kPa, i.e. ±2 kPa relative to the resting pressure within the ear canal. As such, the amplitude of the pressure change applied is limited. As mentioned above, preferably the tympanic cavity pressure is equalized with the ear canal pressure before generation of oscillating pressure within the ear canal. This ensures that pressures generated within the ear canal do not damage the tympanic membrane, which can occur when the difference in pressures of the ear canal and tympanic cavity exceeds 20 kPA, i.e. ±20 kPa.

The maximum deviation from the resting pressure, i.e. the maximum magnitude of the pressure change generated within the ear canal is in embodiments at least 0.5 kPa, i.e. ±0.5 kPa.

In embodiments the oscillating pressure generated in the ear canal has a maximum magnitude (i.e. amplitude) between 0.5 kPa and 4 kPa, i.e. between ±0.5 kPa and ±4 kPa. Such amplitudes allow for maximum deflection of the ear drum whilst restricting any possible damage to the ear drum.

The oscillations are configured to generate a maximal, harmonic fluid movement.

By providing a sinusoidal pressure change it is possible to maximise eardrum movement and therefore, fluid movement in the cochlea to maximise drug distribution along it. The generated pressure amplitude is provided from a resting pressure. As such movement of the eardrum towards the extremities can be maximised.

Generation of purely harmonic pressure avoids signal distortion and generation of harmonics (signals at frequencies 2f, 3f . . . of the main frequency). This restricts stimulation of the cochlea which may deafen users if the pressure oscillations contain higher harmonics.

In embodiments the method comprises generating air pressure oscillations at a frequency in the range of 1 to 20 Hz, more preferably 2 to 20 Hz, still more preferably 4 to 18 Hz, still more preferably 8 to 16 Hz. Oscillations at a frequency in the range of 2 to 10 Hz may be used.

The step of generating oscillating pressure within the ear canal is preferably performed immediately after administration of the substance to the middle ear. The step of generating oscillating pressure within the ear canal may however be performed after a short period of time, preferably up to 15 minutes, more preferably up to 10 minutes, more preferably up to 5 minutes, has elapsed following administration of the substance to the middle ear. In other words, the step of generating oscillating air pressure within the ear canal may be initiated within 15 minutes of, more preferably within 10 minutes of, more preferably within 5 minutes of, most preferably immediately following the completion of the step of administering the substance to the middle ear.

After administration of substances to the middle ear, patients are typically instructed to lie still for a period of about 30 minutes. This period following intratympanic administration is a preferred time for the performance of the oscillating pressure generating steps of the present methods. Preferably the patient is lying down during the performance of the oscillating pressure generation step. Thus, preferably, oscillating air pressure within the ear canal is generated for a duration of from 1 to 30 minutes, more preferably from 5 to 30 minutes, more preferably from 5 to 20 minutes, more preferably from 10 to 20 minutes, most preferably for about 15 minutes. Durations of from 1 to 20 minutes, from 1 to 15 minutes, from 1 to 10 minutes, from 1 to 5 minutes, from 5 to 10 minutes, from 5 to 15 minutes, from 10 to 30 minutes or from 10 to 15 minutes may also be used.

The inventors of the present invention have recognised that, contrary to conventional practice, the generation of oscillating air pressure, particularly when the generated pressure changes are biphasic, causes substances to be driven from the tympanic cavity into the cochlea and along the cochlear spiral by a periodic flow of perilymph. Without wishing to be bound by theory, it is believed that the generation of oscillating air pressure, particularly biphasic pressure changes, within the ear canal causes reciprocated movement of the round and/or oval window membrane and/or stapes, leading to perilymph flow. Using the methods of the present invention, substances deposited in the middle ear, particularly in the tympanic cavity and on the round and oval windows, are able to diffuse successfully into the cochlear perilymph and along the entire cochlea spiral. Using the methods of the invention, the present inventors have demonstrated improved delivery of substances to the inner ear via improved distribution along the cochlear spiral, and even improved delivery to the cochlear apex.

Thus, the present invention also provides a method for inducing or promoting transfer of a substance from the middle ear to the inner ear, the method comprising the steps of:
i) administering the substance to the middle ear; and
ii) generating oscillating air pressure within the ear canal.

The invention also provides a method for inducing or promoting transfer of a substance from the middle ear to the inner ear, wherein the substance has been administered to the middle ear and wherein the method comprises generating oscillating air pressure within the ear canal.

As used herein, “inducing” refers to the initiation of transfer, and “promoting” refers to increasing the amount or rate of transfer as compared to the amount or rate of transfer that occurs without generation of oscillating air pressure within the ear canal.

The invention also provides a method for distributing a substance along the cochlear spiral, the method comprising the steps of:
i) administering the substance to the middle ear; and
ii) generating oscillating air pressure within the ear canal.

The invention also provides a method for distributing a substance along the cochlear spiral, wherein the substance has been administered to the middle ear and wherein the method comprises generating oscillating air pressure within the ear canal.

The invention also provides a method for inducing or promoting periodic flow of perilymph within the inner ear, the method comprising generating oscillating air pressure within the ear canal.

As used herein, the term “periodic flow” refers to the pattern of flow of the perilymph having a repeating nature, which is caused by the oscillating nature of the pressure changes generated in the ear canal.

The definitions, preferred and optional features and embodiments of the disclosed methods of delivering a substance to the inner ear apply mutatis mutandis to each and all of these additional methods of the invention.

Given that the methods of the present invention achieve improved delivery of substances, including therapeutic compounds, to the inner ear, they have significant advantages in the treatment and prevention of inner ear disorders.

Thus, in a further aspect the present invention provides a method of treating or preventing an inner ear disease, the method comprising the steps of:
i) administering a therapeutic compound to the middle ear; and
ii) generating oscillating air pressure within the ear canal.

The present invention also provides a method of treating or preventing an inner ear disease, wherein a therapeutic compound has been administered to the middle ear and wherein the method comprises generating oscillating air pressure within the ear canal.

Alternatively viewed, the present invention provides a therapeutic compound for use in a method of treating or preventing an inner ear disease, wherein said method comprises the steps of:
i) administering the therapeutic compound to the middle ear; and
ii) generating oscillating air pressure within the ear canal.

The present invention also provides a therapeutic compound for use in a method of treating or preventing an inner ear disease, wherein the therapeutic compound has been administered to the middle ear and wherein said method comprises generating oscillating air pressure within the ear canal.

Alternatively viewed, the present invention provides the use of a therapeutic compound in a method of treating or preventing an inner ear disease, wherein said method comprises the steps of:
i) administering the therapeutic compound to the middle ear; and
ii) generating oscillating air pressure within the ear canal.

The present invention also provides the use of a therapeutic compound in a method of treating or preventing an inner ear disease, wherein the therapeutic compound has been administered to the middle ear and wherein said method comprises generating oscillating air pressure within the ear canal.

Alternatively viewed, the present invention provides a therapeutic compound for use in the manufacture of a medicament for use in the treatment or prevention of an inner ear disease, wherein said treatment or prevention comprises the steps of:
i) administering the therapeutic compound to the middle ear; and
ii) generating oscillating air pressure within the ear canal.

As used herein, “treatment” includes any therapeutic application that can benefit a human or non-human animal (e.g. a non-human mammal). Both human and veterinary treatments are within the scope of the present invention, although primarily the invention is aimed at the treatment of humans. Treatment may be in respect of an existing disease or condition or it may be prophylactic. Thus, both treatment and prevention are encompassed by the present invention.

Preferably, in the methods of treatment of the present invention, a pharmaceutically effective amount of therapeutic compound is administered, or has previously been administered, to the middle ear. By “pharmaceutically effective amount” is meant an amount that will lead to the desired pharmacological and/or therapeutic effect, i.e. an amount of the agent which is effective to achieve its intended purpose. While individual patient needs may vary, determination of optimal ranges for effective amounts of the active agent is within the capability of one skilled in the art. Generally, the dosage regimen for treating a disease or condition with any of the compounds described herein is selected in accordance with a variety of factors including the nature of the medical condition and its severity.

The dosage required to achieve the desired activity of the compounds herein described will depend on various factors, such as the compound selected, whether the treatment is therapeutic or prophylactic, and the nature and severity of the disease or condition, etc. Typically, a physician will determine the actual dosage which will be most suitable for an individual subject. The specific dose level and frequency of dosage for any particular patient may be varied and will depend upon factors such as the activity of the specific compound employed, the metabolic stability and length of action of that compound, the age of the patient, the time of administration, and the severity of the particular condition.

The method of the invention is generally applicable given that it enhances the passage of substances in general to the inner ear. Thus, the inner ear disease treated or prevented according to the present invention may be any ear disease.

Preferably the inner ear disease is selected from the group consisting of hearing loss, preferably age-related hearing loss (ARHL), sensorineural hearing loss (SNHL), chemically-induced hearing loss (CIHL), noise-induced hearing loss (NIHL); ototoxicity; Meniere's disease; tinnitus; infection, preferably bacterial, viral or fungal infection; inflammation; autoimmune diseases, preferably immune-mediated cochleovestibular disorders (IMCVDs) and autoimmune inner ear disease (AIED); congenital disease or dysfunction and inner ear vestibular dysfunction.

The therapeutic compounds used to treat inner ear disorders are well-defined and any therapeutic compound may be used in accordance with the present invention. The therapeutic compound is preferably as defined above. The therapeutic compound is preferably present in a pharmaceutical composition as described above, preferably as a liquid, hydrogel or nanovector as described above.

Preferably, the therapeutic compound and inner ear disease are selected from the following:

| Therapeutic | Preferred therapeutic compound | |
|---|---|---|
| Compound | selected from the group consisting of: | Inner ear disease: |
| Corticosteroid | Dexamethasone, methylprednisolone, | SNHL, Menière's disease, AIED, |
| | betamethasone | NIHL, tinnitus |
| Antibiotic | Gentamicin, streptomycin, kanamycin | Menière's disease |
| Prostaglandin | Latanoprost | Menière's disease |
| receptor | | |
| agonist | | |
| Antioxidant | n-Acetyl cysteine, d-methionine, | NIHL |
| | thiourea | |
| Anaesthetic | Lidocaine | Tinnitus |
| n-Methyl-d- | Esketamine, gacyclidine, ifenprodil | Tinnitus |
| aspartate | | |
| receptor | | |
| antagonist | | |
| Antiviral | Cidofovir, ganciclovir | Menière's disease, viral infection, |
| | | preferably CMV infection, SNHL |
| | | due to CMV infection |
| Apoptpsis | e.g. AM-111 peptide (JNK inhibitor) | NIHL |
| inhibitors | | |
| Gamma | LY3056480 | SNHL, ARHL |
| secreatase | | |
| inhibitor | | |
| Neurotrophin | BDNF, neurotrophic factor-3, growth | SNHL |
| | factors | |
| Monoclonal | Infliximab, Golimumab, Anakinra, | AIED |
| Antibody | Rituximab | |
| Nucleic acid | Plasmids, siRNA | SNHL |
| Stem cell | | SNHL |

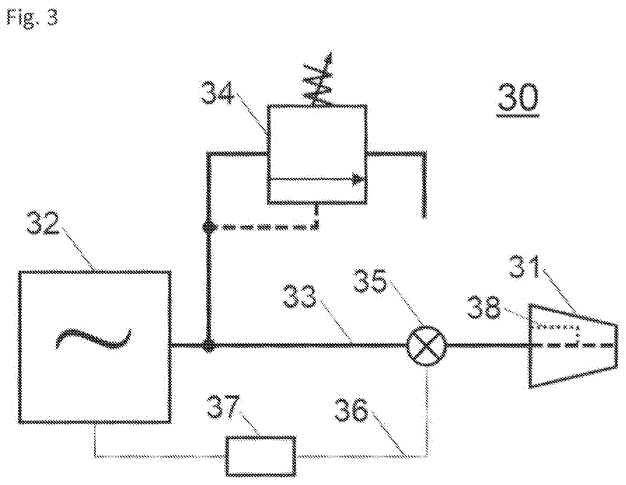

Referring now to FIG. 3, an embodiment of a device for delivering substances into the cochlea is shown. The device 30 comprises an aural probe 31, a pressure oscillator 32, and a tube 33. A fluid passageway is defined in the device 30. The fluid passageway defines a fluid path between the pressure oscillator 32 and an end of the aural probe 31 inserted into a patient's ear. The aural probe 31, pressure oscillator 32, and tube 33 may define, at least in part, the fluid passageway. The device 30 has a pressure relief valve 34.

The aural probe 31 is configured to be inserted into a patient's ear, e.g. in the ear canal, and comprises an aperture through which pressurised air is delivered to the patient's ear. The aural probe 31 comprises an air passageway that extends from a first end to the aperture to a second end opposite the first end, the first end being the end to be inserted into the ear canal. An opening at the first end defines the end of the fluid passageway. The oscillating air pressure being applied to the tympanic membrane of the patient causes reciprocating movements in the tympanic membrane. Pulses of air pressurised at a suitable and desired pressure within a predetermined range are generated or otherwise output by the pressure oscillator 32 at a predetermined frequency. In some embodiments, a pressure pump is provided (not shown) coupled to the pressure oscillator 32 for generating or outputting air pressure oscillations at a predetermined oscillation frequency. The pressure pump, in embodiments, forms part of or forms the pressure oscillator 32. The pressure oscillator, according to present embodiments, preferably comprises a multiplexor, which alternately switches at the predetermined frequency the input to the aural probe between atmospheric pressure (or zero differential pressure across the tympanic membrane) and the pressure pump set to generate air pressure at a predetermined pressure, thus generating pressure pulses at a predetermined oscillation frequency at a predetermined magnitude. In embodiments, the oscillating air pressure is generated at a frequency in the range of 2 to 20 Hz. The oscillating air pressure may be delivered from the pressure oscillator 32 to the aural probe 31 through the tube 33. The tube 33 is made from a flexible material such as rubber and silicone, but other flexible, rigid or semi-flexible materials are contemplated. In embodiments, the tube 33 is coupled to the aural probe 31 via the pressure relief valve 34. The pressure relief valve 34 is configured to allow pressurised air to escape from the tube 33 or the aural probe 31 before reaching the patient's ear, so as to avoid overstimulation of the tympanic membrane due to excessive air pressure, thereby preventing the rupture of the tympanic membrane. The pressure relief valve 34 may be configured to release pressurised air within the tube 33 and/or the aural probe 31 manually by means of a switch, a button or the like, or it may be configured to release pressurised air automatically via a feedback mechanism.

A processor 37 is provided. The processor 37 is part of a control unit configured to control the pressure oscillator 32 and/or the pressure relief valve 34. The control unit comprises a memory. The processor 37 is operable to control the oscillator 32 and/or the pressure relief valve 34 to regulate the pressure in the device and therefore the ear canal. The control unit in some embodiments is part of the pressure oscillator 32.

In some embodiments, a pressure sensor 35 may be provided for measuring the air pressure within the tube 33. The pressure sensor 35 may be configured to continuously monitor the air pressure within the tube 33, or it may be configured to determine the air pressure within the tube 33 intermittently or periodically. The pressure sensor 35 may be configured to determine the instantaneous pressure within the tube 33 or it may be configured to determine a time-averaged pressure within the tube 33 over a period of time, as desired. It is therefore possible to continuously or periodically monitor the air pressure delivered to the patient's ear, and manually adjust the air pressure as required using the pressure oscillator 32 or the pressure relief valve 34 as required.

In further embodiments, a feedback mechanism may be provided, comprising a feedback signal line 36 and the processor 37, between the pressure sensor 35 and the pressure relief valve 34. Signals from the pressure sensor 35 are processed by the processor 37 to determine if the pressure within the tube 33 exceeds a predetermined maximum pressure. If it is determined that the pressure within the tube 33 exceeds the predetermined maximum pressure, the pressure relief valve 34 is activated to release pressurised air from the tube 33. Alternatively or in addition, the feedback signal line 36 may be provided between the pressure sensor 35 and the pressure oscillator 32. A desired air pressure may be set e.g. by a user, and signals from the pressure sensor 35 are processed by the processor 37 to determine if the pressure within the tube 33 is within a predetermined tolerance range of the desired air pressure. When the processor 37 determines that the air pressure within the tube 33 is outside of the tolerance range, the processor 37 causes the pressure oscillator 32 to adjust the output air pressure upwards or downwards as required. The feedback signal line 36 and the processor 37 may be provided as an integrated feedback module. Alternatively, the processor 37 is part of the control unit configured to control the pressure oscillator 32 and/or the pressure relief valve 34. It is therefore possible to continuously monitor and adjust the air pressure delivered to the patient's ear automatically.

In further embodiments, Siegel speculum or pneumatic otoscope (not shown) may be used alternatively or in addition to the aural probe 31. The aural probe 31 may comprise a Siegel speculum or pneumatic otoscope (not shown). A Siegel speculum or pneumatic otoscope allows visualization of tympanic membrane movement within the patient's ear, and is therefore particularly suited for use with patients who are unable to provide adequate verbal feedback during the procedure, for example, in young children or in patients with tympanic membrane desensitized through anaesthesia.

According to the present disclosure, oscillating air pressure in the ear canal leads to oscillating movement of the tympanic membrane, which is transmitted to the cochlear fluid through the middle ear bones. A substance may be pre-installed into the middle ear cavity by any suitable means such as application of the substance on the round and/or oval window membrane, and the substance diffuses into the cochlea through the round and oval window membranes. The reciprocating movements of the cochlear round and oval window membranes and associated periodic fluid movement enable improved distribution of the substance along a substantial or even the entire length of the cochlear spiral. Without being constrained by theory, the process may be due to the combined effects of narrowing of the cochlear duct and chaotic advection.

### Application to Human Patients

In an embodiment implementing the method and/or device 30 on human patients, drug solutions may be loaded into the middle ear of a patient through the tympanic membrane or through the Eustachian tube. The patient's head may be placed in such a position that avoids the escape of the drug solutions through the Eustachian tube.

In the present embodiment, the aural probe 31 is sealed in the ear canal of the patient. An air tight seal with the canal may be provided in routine clinical use through a range of aural probes of different shapes and sizes to allow for the natural variations in shapes and sizes of the ears of different patients. In some embodiments, this may be achieved using a range of disposable tips in conjunction with a common probe base for convenience and/or hygienic reasons.

Optionally, since the aural probe 31 is to be sealed in the ear canal of the patient, it may be desirable to provide a closable auxiliary air passageway 38 through the probe 31 whereby the pressure on either side of the tympanic membrane may be equalised after placement of the probe 31 in the patient's ear and before the operation of the device 30. The auxiliary air passageway 38 is arranged to extend from an interior surface of the air passageway of the aural probe 31 to an exterior surface of the aural probe at the second end, and preferably comprises a closable valve for opening or closing the auxiliary air passageway.

In the present embodiment, oscillating air pressure at a frequency of 2 to 20 Hz is generated by the pressure oscillator 32 and delivered to the aural probe 31. The oscillating air pressure is delivered through the flexible tube 33. A maximum oscillation frequency may be set to avoid stimulation of the sensory apparatus of the cochlea and potentially inflicting permanent hearing loss. For example, the maximum frequency of pressure oscillation may be set at 20 Hz which approximately corresponds to the cut-off frequency of the human helicotrema.

Stimulation by pressure oscillation is preferably start with low peak pressure values, e.g. 0.5 kPa, preferably from zero peak pressure values, and gradually increasing the air pressure as required while ensuring that the patient is not experiencing any unpleasant sensations or pain. Siegel speculum or pneumatic otoscope may be used alternatively or in addition to the aural probe 31 to allow visualization of tympanic membrane movement, especially in patients who are unable to provide adequate feedback during the procedure, for example, in young children or in patients with tympanic membrane desensitized through anaesthesia.

At a pressure of about 20 kPa or above, minor, moderate, to major eardrum ruptures may occur. It may therefore be desirable to pre-set the pressure relief valve 34 at a maximum pressure of 20 kPa or below to avoid overstimulation and rupture of the tympanic membrane by excessively high air pressure.

Setting the pressure oscillator 32 to displace the same volume of air does not guarantee a constant pressure at the tympanic membrane. During the procedure, pressure at the tympanic membrane may change as a result of movement of the aural probe 31, changes in the ear canal shape, changes in the mechanical impedance of the middle ear, etc. It may therefore be desirable to set the pressure sensor 35 to measure the instantaneous pressure in the tube 33 periodically to continuously monitor the actual pressure at the tympanic membrane, and to continuously adjust the tube pressure to a desired value either automatically through the feedback signal line 36 or using the manual control of the pressure oscillator 32.

Further embodiments, optional and alternative features have been contemplated. For example, while preferred embodiments have been described in relation to pressure stimulations through the ear canal, pressure changes may alternatively or additionally be generated through the Eustachian tube, or differentially through the Eustachian tube and ear canal. In patients whose tympanic membranes are damaged, then the fluid movement and distribution of substances within the cochlea may be achieved through direct movement of the ossicular chain or its parts, or round window membrane by means of mechanical probes. Embodiments of the device may be a stand-alone unit or as part of an ENT (ear, nose and throat) workstation/treatment unit, e.g. including powered suction systems and/or compressed air systems, which may be incorporated to provide pressure oscillations.

Although the present invention has been described with reference to preferred embodiments, it will be understood by those skilled in the art that various changes in form and detail may be made without departing from the scope of the invention as set forth in the accompanying claims.

The invention will now be further described in the following non-limiting Examples:

### EXAMPLES

### Animals

Pigmented guinea pigs of similar weight (350-360 g) were anaesthetised with the neurolept anaesthetic technique (0.06 mg/kg body weight atropine sulphate s.c., 30 mg/kg pentobarbitone i.p., 500 μl/kg Hypnorm i.m.). Additional injections of Hypnorm were given every 40 minutes. Additional doses of pentobarbitone were administered as needed to maintain a non-reflexive state. The heart rate was monitored with a pair of skin electrodes placed on both sides of the thorax. The animals were tracheotomized and artificially respired, and their core temperature was maintained at 38° C. with a heating blanket and a heated head holder.

### Signal Generation and Recording

The middle ear cavity of the ear used for the measurements was opened to reveal the round window. Compound action potentials of the auditory nerve were measured from the cochlear bony ridge in the proximity of the round window membrane using Teflon-coated silver wire coupled to laboratory designed and built extracellular amplifier (James Hartley). Thresholds of the N1 peak of the compound action potentials were estimated visually using 10 ms tone stimuli at a repetition rate of 10 Hz.

For acoustic stimulation sound was delivered to the tympanic membrane by a closed acoustic system comprising two Bruel and Kjaer 4134½″ microphones for delivering tones and a single Bruel and Kjaer 4133½″ microphone for monitoring sound pressure at the tympanum. The microphones were coupled to the ear canal via 1 cm long, 4 mm diameter tubes to a conical speculum, the 1 mm diameter opening of which was placed about 1 mm from the tympanum. The closed sound system was calibrated in situ for frequencies between 1 and 50 kHz. Known sound pressure levels were expressed in dB SPL re 2×10⁻⁵ Pa. All acoustic stimuli in this work were shaped with raised cosines of 0.5 ms duration at the beginning and at the end of stimulation. White noise for acoustical calibration and tone sequences for auditory stimulation were synthesised by a Data Translation 3010 board at 250 kHz sampling rate and delivered to the microphones through low-pass filters (100 kHz cut-off frequency). Signals from the acoustic measuring amplifier (James Hartley) were digitised at 250 kHz sampling rate using the same board and averaged in the time domain. Experimental control, data acquisition and data analysis were performed using a PC with programmes written in MATLAB.

5 μl of 100 mM sodium salicylate solution in Hanks' Balanced Salt Solution were placed on the round window using pipettes. The solution was removed from the round window using paper wicks to observe the wash out effect.

Experiments were made on deeply anaesthetised guinea pigs which, for the purpose of the experiments, have cochlear structure similar to humans. All procedures involving animals were performed in accordance with UK Home Office regulations with approval from the local ethics committee.

Sodium salicylate is a model drug which diffuses through the round window membrane and has well-characterized physiological effects. One of these effects is elevation of threshold responses of the auditory nerve to a pure tone stimulation, i.e. a higher sound pressure of pure tones in the ear canal is required to cause responses in the auditory nerve when salicylate spreads along the cochlea and affects its function. The cochlea is tonotopically organized. Namely, high-frequency tones produce the largest response at the base and low-frequency tones at the apex of the cochlea (Greenwood, 1990 J. Acoust. Soc. Am. 87, 2592-2605). Hence, sweeping tone frequency, it is possible to study salicylate effect at different cochlear places. Spatial presentation of frequencies along the cochlea is not linear but logarithmic and higher frequencies are represented in denser pattern near the cochlear base.

In the first experiments, 5 μl of 100 mM salicylate solution was placed on the round window of a guinea pig and left to diffuse passively into the cochlea.

In the second experiments, the placement of the same volume of salicylate solution on the round window was followed by stimulation of the ear with oscillating air pressure in the ear canal during a 5-minute period with a consecutive 5-minute period without oscillating air pressure stimulation when the auditory nerve thresholds were measured. The cycle of pressure-stimulation/no-pressure-stimulation was repeated 8 times until elevation of the auditory nerve thresholds reached saturation. The oscillating air pressure was generated by a device of the invention, with the aural probe (31) being inserted into the ear canal. Low-frequency air pressure oscillations at frequency 4 Hz caused large-scale oscillations of the eardrum which were transmitted to the stapes through the middle ear bones. Stapes oscillations (about 100 μm peak-to-peak) caused perilymph oscillations and reciprocated movement of the round window membrane. Pure tone sound pressure level which caused threshold response of the auditory nerve was measured in both experiments using sound system inserted into the ear canal.

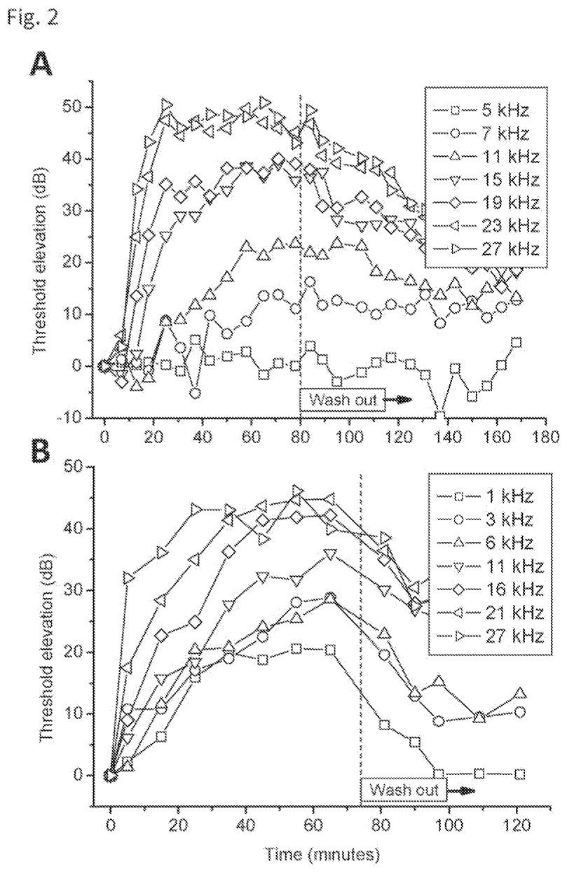

As shown in FIG. 2A, in the first experiment, no effect of salicylate on the auditory nerve threshold was observed for tone frequencies lower than 5 kHz, which corresponds to approximately the apical 55% of cochlear length. Thus passive diffusion was demonstrated not to lead to substantial salicylate distribution along the cochlear spiral.

In contrast, as shown in FIG. 2B, the salicylate effect was observed after 5 to 10 minutes of oscillating pressure generation, even for 1 kHz tone which corresponds to the apical 25% of the cochlear spiral. The effect saturated after 15-20 minutes of the total pressure stimulation time (without taking into account the 5-minute no-pressure-stimulation periods), within which the drug could be evenly distributed along the entire cochlea. No threshold elevation was recorded in control experiments during pressure stimulation without salicylate application. Hence, the observed threshold elevation is not because of any damage inflicted by the pressure stimulation but entirely due to salicylate action. This demonstrates that the generation of oscillating pressure within the ear canal in accordance with the present invention results in improved distribution of substances along the length of the cochlear spiral. Partial recovery of the compound action potential thresholds after washing out of salicylate from the round window (FIG. 2) serves as additional confirmation that the integrity of the sensory cells was preserved.

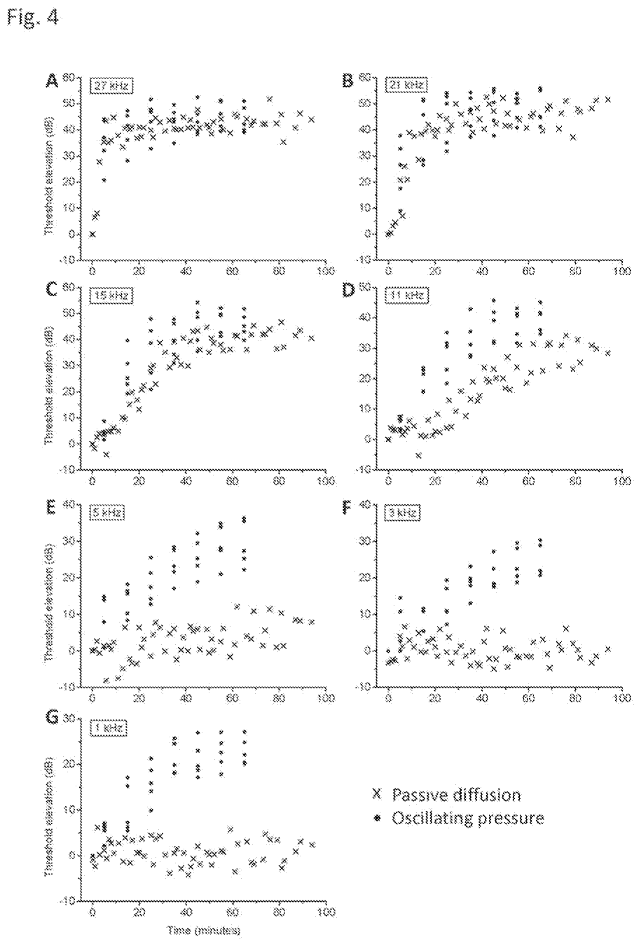

Pooled data for differing elevation of the auditory nerve threshold responses following no treatment (passive diffusion, crosses) or generation of oscillating pressure within the ear canal (oscillating pressure, circles) in response to pure tones of frequency 27 kHz (A), 21 kHz (B), 15 kHz (C), 11 kHz (D), 5 kHz (E), 3 kHz (F) and 1 kHz (G) is shown in FIG. 4. The cochlea is tonotopically organized. High-frequency tones produce the largest response at the base and low-frequency tones at the apex of the cochlea (Greenwood, 1990 J. Acoust. Soc. Am. 87, 2592-2605). FIG. 4 shows that the threshold response of the cochlear to pure tones of all frequencies, particularly low frequencies corresponding to the apical portion of the cochlear spiral, is increased by salicylate following the generation of oscillating pressure within the ear canal to a far greater extent than when salicylate is merely allowed to diffuse passively into the cochlea. This demonstrates that the generation of oscillating pressure within the ear canal achieves markedly improved distribution of substances along the full length of the cochlear spiral, even at the apical portion.

1. A device for assisting in distributing a substance in the inner ear, comprising:
a pressure oscillator;
an aural probe having an end insertable in an ear canal; and
a fluid passageway fluidly communicating the pressure oscillator and the end of the aural probe insertable in the ear canal;
wherein the pressure oscillator is configured to generate an oscillating fluid pressure change in the fluid passageway.

2. The device according to claim 1, wherein the pressure oscillator is configured to generate both positive and negative pressures in the fluid passageway.

3. The device according to claim 1, wherein the pressure oscillator is configured to generate a biphasic oscillating pressure change in the fluid passageway.

4. The device according to claim 1 wherein a predetermined maximum pressure amplitude is less than 20 kPa, optionally less than 4 kPa and, optionally, greater than 0.5 kPa and, optionally, less than 2 kPa.

5. The device according to claim 1 wherein the pressure oscillator is configured to generate air pressure oscillations at a frequency in the range of 1 to 20 Hz, and optionally 2 to 20 Hz, and optionally 4 to 18 Hz, and optionally 8 to 16 Hz.

6. The device according to claim 1, comprising a pressure relief valve communicating with the fluid passageway and configured to open when a pressure within the fluid passageway reaches a predetermined maximum positive and/or negative operating pressure.

7. A device for delivering a substance in the inner ear, comprising:
a pressure oscillator configured to generate pressurised air at a predetermined oscillation frequency;
an aural probe comprising an air passageway extending from a first end of the aural probe to a second end of the aural probe, the first end being the end to be inserted into the ear canal;
a fluid passageway between the pressure oscillator and the first end of the aural probe; and
a pressure relief valve disposed at the fluid passageway configured to release a portion or the generated pressurised air from within the fluid passageway when a pressure within the tube and/or the aural probe reaches a predetermined maximum pressure.

8. The device of claim 7, comprising a tube arranged to couple the second end of the aural probe to the pressure oscillator.

9. The device according to claim 7, further comprising a pressure sensor coupled to the fluid passageway configured to determine an air pressure within the fluid passageway.

10. A method of operating a device for assisting in distributing a substance in the inner ear, the device including an aural probe having an end insertable in an ear canal, the method comprising generating an oscillating fluid pressure change in a fluid passageway of the device to supply both positive and negative pressures at an end of an aural probe.

11. A method for assisting in the delivery of a substance to the inner ear, the method comprising the steps of:
i) administering the substance to the middle ear; and
ii) generating oscillating air pressure within the ear canal.

12. A method for assisting in the delivery of a substance to the inner ear, wherein the substance has been administered to the middle ear and wherein the method comprises generating oscillating air pressure within the ear canal.

13. The method of claim 11, wherein delivery is to the cochlea, preferably along the cochlear spiral.

14. A therapeutic compound for use in a method of treating or preventing an inner ear disease, wherein said method comprises the steps of:
i) administering the therapeutic compound to the middle ear; and
ii) generating oscillating air pressure within the ear canal.

15. A therapeutic compound for use in a method of treating or preventing an inner ear disease, wherein the therapeutic compound has been administered to the middle ear and wherein said method comprises generating oscillating air pressure within the ear canal.

16. The therapeutic compound for use of claim 14, wherein the inner ear disease is selected from the group consisting of hearing loss, ototoxicity, Menière's disease, tinnitus, infection, inflammation; an autoimmune disease, a congenital disease or dysfunction and inner ear vestibular dysfunction, preferably, wherein the hearing loss is age-related hearing loss (ARHL), sensorineural hearing loss (SNHL), chemically-induced hearing loss (CIHL) or noise-induced hearing loss (NIHL), wherein the infection is bacterial, viral or fungal infection, or wherein the autoimmune disease is an immune-mediated cochleovestibular disorder (IMCVD) or autoimmune inner ear disease (AIED).

17. The therapeutic compound for use of claim 14, wherein the therapeutic compound is selected from the group consisting of a protein, a peptide, a non-protein drug, a small chemical molecule, a carbohydrate, a lipid, a lipopolysaccharide, a nucleic acid, a viral vector and a stem cell, preferably wherein the therapeutic compound is selected from the group consisting of a steroid, an antibiotic, an antioxidant, an anaesthetic, an n-Methyl-d-aspartate receptor antagonist, an antiviral agent, an antifungal agent, an apoptosis inhibitor, an enzyme inhibitor, a neurotrophic or neuroprotective agent, an antibody, a nucleic acid, a viral vector and a stem cell.

18. The method or therapeutic compound for claim 11, wherein the step of generating oscillating air pressure within the ear canal comprises inserting an aural probe into the ear canal, optionally equalizing the air pressure within the ear canal and the air pressure within the tympanic cavity, and then generating oscillating air pressure within the ear canal by operating an oscillating pressure generating device coupled to said aural probe.

19. The method or therapeutic compound for use of claim 18, wherein said oscillating pressure generating device comprises a pressure pump being coupled to a pressure oscillator configured to output pressurized air at a predetermined oscillation frequency.

20. The method or therapeutic compound for use of claim 11, wherein the oscillating pressure generated in the ear canal has a maximum magnitude between 0.5 kPa and 20 kPa, and optionally between 0.5 kPa and 4 kPa, and optionally between 0.5 kPa and 2 kPa, relative to the resting pressure within the ear canal.

21. The method or therapeutic compound for use of claim 11, wherein the method comprises generating air pressure oscillations at a frequency in the range of 2 to 20 Hz.

22. The method or therapeutic compound for use of claim 11, wherein the step of generating oscillating air pressure within the ear canal comprises generating biphasic oscillating air pressure within the ear canal.

23. (canceled)
